(19) United States
(12) Patent Application Publication
(10) Pub. No.: US 2019/0022227 A1
(43) Pub. Date: Jan. 24, 2019

(54) TOPICAL COMPOSITION COMPRISING GLYCEROL MONOLAURATE

(71) Applicant: Regents of the University of Minnesota, Minneapolis, MN (US)

(72) Inventors: Jayanth Panyam, Minneapolis, MN (US); Ameya Kirtane, Minneapolis, MN (US); Ashley Haase, Minneapolis, MN (US)

(21) Appl. No.: 15/967,844

(22) Filed: May 1, 2018

Related U.S. Application Data
(60) Provisional application No. 62/500,953, filed on May 3, 2017.

(51) Int. Cl. A61K 47/10; A61K 9/00; A61K 31/717; A61P 31/00

(52) U.S. Cl. CPC A61K 47/10; A61P 31/00; A61K 31/717; A61K 9/0034

(57) ABSTRACT

Certain embodiments of the invention provide a pharmaceutical composition suitable for topical administration that comprises more than about 5% (w/w) glycerol monolaurate (GML), as well as methods of preparing and using such compositions.

### CROSS REFERENCE TO RELATED APPLICATIONS

This application claims the benefit of priority of U.S. Provisional Application Ser. No. 62/500,953 filed May 3, 2017, the entire contents of which are incorporated herein by reference.

### STATEMENT AS TO RIGHTS TO INVENTIONS MADE UNDER FEDERALLY SPONSORED RESEARCH AND DEVELOPMENT

This invention was made with Government support under AI071976 awarded by the National Institutes of Health. The Government has certain rights in the invention.

### BACKGROUND

Human immunodeficiency virus (HIV) infection remains a major epidemic with ˜2.1 million new cases and ˜1.5 million acquired immunodeficiency syndrome (AIDS)—related deaths reported in 2013 (Centers for Disease Control and Prevention 2015 HIV Surveillance Report: Diagnoses of HIV Infection and AIDS in the United States and Dependent Areas, 2013 2015 www.cdc.gov/hiv/statistics/basics/). Women are disproportionately burdened by HIV in many regions (European Study Group on Heterosexual Transmission of HIV. 1992. Comparison of female to male and male to female transmission of HIV in 563 stable couples. BMJ 304(6830):809-813; Krishnan et al. 2008. Ann N Y Acad Sci 1136:101-110), including in Sub-Saharan Africa, which is home to over 70% of the world's HIV infected population (Morgan D, Whitworth J 2001. Nat Med 7(2):143-145). Therefore, effective female controlled HIV prevention options are clearly needed. Although antiretroviral (ARV) containing vaginal products for HIV prevention have had encouraging results (Abdool et al. 2010. Science 329(5996):1168-1174; Baeten et al. 2016. Use of a Vaginal Ring Containing Dapivirine for HIV-1 Prevention in Women. N Engl J Med. 375:2121-2132; Nel et al. 2009. J Acquir Immune Defic Syndr 51(4):416-423), adherence to these products has proven to be a major issue (Baeten et al. 2016. Use of a Vaginal Ring Containing Dapivirine for HIV-1 Prevention in Women. N Engl J Med. 375:2121-2132; Marrazzo et al. 2015. N Engl J Med 372(6):509-518; Van Damme et al. 2012. N Engl J Med 367(5):411-422). The presence of pre-existing inflammation in the female reproductive tract (FRT) has also been shown to decrease the efficacy of vaginal microbicides (Masson et al. 2015. Clin Infect Dis 61(2):260-269). Therefore, a vaginal product that is highly acceptable to women and also capable of modulating inflammation in the FRT has the potential to be more effective than previously studied formulations.

Glycerol monolaurate (GML), a fatty acid monoester commonly used in the cosmetic and food industries, has been shown to block the expression of pro-inflammatory cytokines and chemokines expressed in the FRT in response to HIV (Li et al. 2009. Nature 458(7241):1034-1038; Schlievert et al. 2008. Antimicrob Agents Chemother 52(12):4448-4454). Blocking these signaling pathways inhibits the recruitment of CD4⁺ target cells to the site of initial infection, which in turn prevents the local expansion of HIV that ultimately leads to systemic dissemination of infection (Li et al. 2009. Nature 458(7241):1034-1038; Roddy et al. 1998. N Engl J Med 339(8):504-510). When applied vaginally, a gel containing 5% GML has been shown to protect against repeated high dose vaginal challenge of simian immunodeficiency virus (SIV) in rhesus macaques (Li et al. 2009. Nature 458(7241):1034-1038). Furthermore, the vaginal delivery of GML has been shown to be safe for repeated use and to have minor effects on the vaginal flora (Schlievert et al. 2008. Antimicrob Agents Chemother 52(12):4448-4454; Strandberg et al. 2010. Antimicrob Agents Chemother 54(2):597-601).

While its mechanism of action, efficacy and safety profile makes GML an attractive microbicide candidate, the physicochemical properties of the gel used in studies to date are not optimal. Because of GML's hydrophobicity, KY warming gel has been used as the vehicle rather than hydroxyethyl cellulose (HEC)-based aqueous gel, the preferred vehicle for vaginal microbicides (Tien et al. 2005. AIDS Res Hum Retroviruses 21(10):845-853). Furthermore, given the solubility issues of GML, the maximum concentration of the drug achievable in the original formulation was only 5%. Higher doses of GML are expected to be even more effective in preventing HIV transmission. Finally, the original 5% GML/KY warming gel formulation has very low viscosity and is associated with leakage when applied vaginally, a characteristic that may impact acceptability (McConville et al. 2014. Clin Med Insights Womens Health 7:1-8). Accordingly, there is a need for new formulations comprising GML (e.g., topical formulations suitable for vaginal administration).

### SUMMARY

Certain embodiments of the invention provide a pharmaceutical composition suitable for topical administration that comprises more than 5% (w/w) glycerol monolaurate (GML).

Certain embodiments of the invention provide a pharmaceutical composition suitable for topical administration that comprises

a) more than about 5% (w/w) glycerol monolaurate (GML);

b) about 2% (w/w) polysorbate 80;

c) about 1% (w/w) hydroxyethyl cellulose (HEC);

d) about 20% (w/w) glycerol; and

e) water.

The invention also provides processes disclosed herein that are useful for preparing a composition described herein.

Thus, certain embodiments of the invention provide a method of preparing a glycerol monolaurate (GML) cream, comprising:

combining GML and a surfactant to provide a first mixture;

combining a thickening agent, an alcohol and water to provide a second mixture; and

combining the first and second mixtures under conditions suitable to provide a cream.

Certain embodiments of the invention provide a method to deliver glycerol monolaurate (GML) to a mammal in need thereof, comprising topically administering an effective amount of a pharmaceutical composition as described herein to the mammal.

Certain embodiments of the invention provide a pharmaceutical composition as described herein for use in medical therapy.

Certain embodiments of the invention provide a pharmaceutical composition as described herein for reducing the likelihood of human immunodeficiency virus (HIV) transmission.

Certain embodiments of the invention provide the use of a pharmaceutical composition as described herein to prepare a medicament for reducing the likelihood of human immunodeficiency virus (HIV) transmission in a mammal.

Certain embodiments of the invention provide a kit comprising a pharmaceutical composition as described herein and instructions for administering an effective amount of the pharmaceutical composition to a mammal in need thereof to reduce the likelihood of human immunodeficiency virus (HIV) transmission.

### BRIEF DESCRIPTION OF THE FIGURES

FIG. 1. Time line of treatment administration, colposcopy, and swab and biopsy collection. Twelve rhesus macaques were randomly assigned to four treatment groups: HEC/Tween 80, 5% GML, 15% GML and 35% GML. Animals were treated with one dose of the various formulations each day for 12 weeks. Vaginal swabs were collected at various time intervals for analysis of cytokine levels, vaginal flora and drug concentration. Tissue biopsies were taken for analysis of drug concentration.

FIGS. 2A-B. Analysis of viscosity of GML creams. GML creams with different concentrations of (A) GML and (B) HEC were prepared and their viscosity was measured using a parallel plate viscometer. A curve describing the power law was fitted to the data. The flow consistency index increased with an increase in concentration of GML and HEC.

FIGS. 3A-C. Vaginal concentrations of GML in rhesus macaques following local administration. Rhesus macaques were treated with three formulations of GML cream containing 5, 15 and 35% GML for 12 weeks. (A) Vaginal swabs and (B) tissue biopsies were collected at various times during the treatment period and drug levels were assessed using GC/MS. Data represented as mean±S.D., n=3, *indicates p<0.05, 35% vs. 5%, one-way ANOVA, post-hoc Bonferroni test. (C) Drug concentrations in vaginal swabs after the completion of 12-week treatment period were measured to determine elimination kinetics of GML. Data is represented as an average of mean±S.D., n=3.

FIGS. 4A-B. Evaluation of cytokine levels in CVF. The concentrations of (A) IL-8 and (B) MIP3α in CVF were analyzed using ELISA. In (A) data is represented as mean±S.D., n=3, *indicates p<0.05, HEC/Tween 80 vs. each treatment group, one-way ANOVA, post-hoc Bonferroni test. In (B) each line indicates a single animal, for several animals levels of MIP3α were below the lower limit of quantification.

FIG. 5. Effect of GML cream on vaginal lactobacilli. CVF was collected using on swabs and then cultured on chocolate agar plates, and the number of lactobacilli colonies were counted. Data represented mean±S.D., n=3. *indicates p<0.05, one-way ANOVA. Post-hoc Bonferroni tests showed that differences between placebo and treatment groups were statistically insignificant.

FIGS. 6A-F. Effect of GML on vaginal microfloral. CVF was collected using on swabs and then cultured on chocolate agar plates, and the number of colonies were counted. Data represented mean±S.D., n=3. *indicates p<0.05, placebo vs. 35% GML, one-way ANOVA, post-hoc Bonferroni.

### DETAILED DESCRIPTION

### Compositions

Certain embodiments of the invention provide a pharmaceutical composition suitable for topical administration that comprises more than about 5% (w/w) glycerol monolaurate (GML).

As used herein “w/w”, refers to the weight percent of the named component to the total weight of the composition (named component plus all other components).

In certain embodiments, the pharmaceutical composition comprises more than 5% (w/w) GML. In certain embodiments, the pharmaceutical composition comprises at least about 10% (w/w) GML. In certain embodiments, the pharmaceutical composition comprises at least about 15% (w/w) GML. In certain embodiments, the pharmaceutical composition comprises at least about 20% (w/w) GML. In certain embodiments, the pharmaceutical composition comprises at least about 25% (w/w) GML. In certain embodiments, the pharmaceutical composition comprises at least about 30% (w/w) GML. In certain embodiments, the pharmaceutical composition comprises at least about 35% (w/w) GML. In certain embodiments, the pharmaceutical composition comprises at least about 40% (w/w) GML. In certain embodiments, the pharmaceutical composition comprises at least about 45% (w/w) GML. In certain embodiments, the pharmaceutical composition comprises at least about 50% (w/w) GML.

In certain embodiments, the pharmaceutical composition comprises between about 6% to about 50% (w/w) GML. In certain embodiments, the pharmaceutical composition comprises between about 6% to about 45% (w/w) GML. In certain embodiments, the pharmaceutical composition comprises between about 6% to about 40% (w/w) GML. In certain embodiments, the pharmaceutical composition comprises between about 6% to about 35% (w/w) GML. In certain embodiments, the pharmaceutical composition comprises between about 6% to about 30% (w/w) GML. In certain embodiments, the pharmaceutical composition comprises between about 6% to about 25% (w/w) GML. In certain embodiments, the pharmaceutical composition comprises between about 6% to about 20% (w/w) GML. In certain embodiments, the pharmaceutical composition comprises between about 6% to about 15% (w/w) GML.

In certain embodiments, the pharmaceutical composition further comprises a thickening agent. As used herein, a “thickening agent” includes compounds suitable for increasing the viscosity of a liquid. In certain embodiments, the thickener improves the suspension of other components of the pharmaceutical composition. In certain embodiments, the thickening agent is a gelling agent (i.e., capable of forming an aqueous gel). In certain embodiments, the thickening agent is a polymer. In certain embodiments, the thickening agent is a water-soluble or water-swellable polymer. In certain embodiments, the thickening agent is a cellulose based polymer. In certain embodiments, the thickening agent is hydroxyethyl cellulose (HEC). In certain embodiments, the thickening agent is hydroxypropyl methylcellulose (HPMC). In certain embodiments, the thickening agent is carbopol. In certain embodiments, the thickening agent is alginate.

In certain embodiments, the pharmaceutical composition comprises between about 0.05% to about 5% (w/w) thickening agent. In certain embodiments, the pharmaceutical composition comprises between about 0.1% to about 5% (w/w) thickening agent. In certain embodiments, the pharmaceutical composition comprises between about 0.1% to about 4% (w/w) thickening agent. In certain embodiments, the pharmaceutical composition comprises between about 0.1% to about 3% (w/w) thickening agent. In certain embodiments, the pharmaceutical composition comprises between about 0.5% to about 2.5% (w/w) thickening agent. In certain embodiments, the pharmaceutical composition comprises between about 0.5% to about 1.5% (w/w) thickening agent. In certain embodiments, the pharmaceutical composition comprises between about 0.5% to about 2.5% (w/w) thickening agent. In certain embodiments, the pharmaceutical composition comprises between about 0.5% to about 2.0% (w/w) thickening agent. In certain embodiments, the pharmaceutical composition comprises between about 0.5% to about 1.5% (w/w) thickening agent. In certain embodiments, the pharmaceutical composition comprises about 1% (w/w) thickening agent.

In certain embodiments, the pharmaceutical composition comprises less than about 5% (w/w) thickening agent. In certain embodiments, the pharmaceutical composition comprises less than about 4% (w/w) thickening agent. In certain embodiments, the pharmaceutical composition comprises less than about 3% (w/w) thickening agent. In certain embodiments, the pharmaceutical composition comprises less than about 2% (w/w) thickening agent. In certain embodiments, the pharmaceutical composition comprises less than about 1% (w/w) thickening agent.

In certain embodiments, the pharmaceutical composition comprises at least about 0.05% (w/w) thickening agent. In certain embodiments, the pharmaceutical composition comprises at least about 0.1% (w/w) thickening agent. In certain embodiments, the pharmaceutical composition comprises at least about 0.5% (w/w) thickening agent. In certain embodiments, the pharmaceutical composition comprises at least about 1% (w/w) thickening agent. In certain embodiments, the pharmaceutical composition comprises at least about 1.5% (w/w) thickening agent. In certain embodiments, the pharmaceutical composition comprises at least about 2% (w/w) thickening agent. In certain embodiments, the pharmaceutical composition comprises at least about 3% (w/w) thickening agent. In certain embodiments, the pharmaceutical composition comprises at least about 4% (w/w) thickening agent.

In certain embodiments, the pharmaceutical composition further comprises a surfactant. As used herein, the term “surfactant” includes detergents, wetting agents, emulsifiers, foaming agents and dispersants, which are suitable to solubilize and/or disperse a hydrophobic molecule (e.g., GML) and do not have unwanted side effects associated with their use (e.g., it is non-toxic). In certain embodiments, the surfactant is polysorbate 80 (i.e., Tween 80), polysorbate 20 (i.e., Tween 20), sodium lauryl sulfate, cetyltrimethylammonium bromide, a pluronic, a Brij surfactant or a sorbitan ester.

In certain embodiments, the pharmaceutical composition comprises between about 1% to about 5% (w/w) surfactant. In certain embodiments, the pharmaceutical composition comprises between about 1% to about 4% (w/w) surfactant. In certain embodiments, the pharmaceutical composition comprises between about 1% to about 3% (w/w) surfactant. In certain embodiments, the pharmaceutical composition comprises between about 1% to about 2.5% (w/w) surfactant. In certain embodiments, the pharmaceutical composition comprises between about 1.5% to about 2.5% (w/w) surfactant. In certain embodiments, the pharmaceutical composition comprises about 2% (w/w) surfactant.

In certain embodiments, the pharmaceutical composition comprises at least about 0.5% (w/w) surfactant. In certain embodiments, the pharmaceutical composition comprises at least about 1% (w/w) surfactant. In certain embodiments, the pharmaceutical composition comprises at least about 1.5% (w/w) surfactant. In certain embodiments, the pharmaceutical composition comprises at least about 2% (w/w) surfactant. In certain embodiments, the pharmaceutical composition comprises at least about 3% (w/w) surfactant. In certain embodiments, the pharmaceutical composition comprises at least about 4% (w/w) surfactant.

In certain embodiments, the pharmaceutical composition comprises less than about 5% (w/w) surfactant. In certain embodiments, the pharmaceutical composition comprises less than about 4% (w/w) surfactant. In certain embodiments, the pharmaceutical composition comprises less than about 3% (w/w) surfactant. In certain embodiments, the pharmaceutical composition comprises less than about 2.5% (w/w) surfactant. In certain embodiments, the pharmaceutical composition comprises less than about 2% (w/w) surfactant.

In certain embodiments, the pharmaceutical composition further comprises an alcohol. In certain embodiments, the alcohol is isopropyl alcohol. In certain embodiments, the alcohol is a polyol, for example, a diol such as propylene glycol, or a triol such as glycerol. In certain embodiments the alcohol functions to increase the viscosity of the composition. In certain embodiments, the alcohol functions as a humectant. In certain embodiments, the alcohol is glycerol.

In certain embodiments, the pharmaceutical composition comprises between about 1% to about 40% (w/w) alcohol. In certain embodiments, the pharmaceutical composition comprises between about 10% to about 30% (w/w) alcohol. In certain embodiments, the pharmaceutical composition comprises between about 15% to about 25% (w/w) alcohol. In certain embodiments, the pharmaceutical composition comprises about 20% (w/w) alcohol.

In certain embodiments, the pharmaceutical composition comprises at least about 10% (w/w) alcohol. In certain embodiments, the pharmaceutical composition comprises at least about 15% (w/w) alcohol. In certain embodiments, the pharmaceutical composition comprises at least about 20% (w/w) alcohol. In certain embodiments, the pharmaceutical composition comprises at least about 30% (w/w) alcohol. In certain embodiments, the pharmaceutical composition comprises at least about 40% (w/w) alcohol.

In certain embodiments, the pharmaceutical composition comprises less than about 40% (w/w) alcohol. In certain embodiments, the pharmaceutical composition comprises less than about 35% (w/w) alcohol. In certain embodiments, the pharmaceutical composition comprises less than about 30% (w/w) alcohol. In certain embodiments, the pharmaceutical composition comprises less than about 25% (w/w) alcohol. In certain embodiments, the pharmaceutical composition comprises less than about 20% (w/w) alcohol. In certain embodiments, the pharmaceutical composition comprises less than about 15% (w/w) alcohol.

In certain embodiments, the pharmaceutical composition further comprises water. In certain embodiments, the water comprises pH modifiers, such a buffering agents.

In certain embodiments, the pharmaceutical composition further comprises a mucoadhesive excipient. As used herein, the term “mucoadhesive excipient” refers to excipients that can interact with the mucous layer associated with biological tissue. Examples include, but are not limited to, pectin, sodium alginate, carboxymethyl cellulose, carbopol, gelatin, collagen and dextran. Thus, in certain embodiments, the mucoadhesive excipient is pectin, sodium alginate, carboxymethyl cellulose, carbopol, gelatin, collagen or dextran.

Certain embodiments of the invention provide a pharmaceutical composition suitable for topical administration that comprises more than about 5% (w/w) glycerol monolaurate (GML), a surfactant, a thickening agent, an alcohol and water.

Certain embodiments of the invention provide a pharmaceutical composition suitable for topical administration that comprises more than about 5% (w/w) glycerol monolaurate (GML), polysorbate 80, hydroxyethyl cellulose, glycerol and water.

Certain embodiments of the invention provide a pharmaceutical composition suitable for topical administration that consists essentially of more than about 5% (w/w) glycerol monolaurate (GML), polysorbate 80, hydroxyethyl cellulose, glycerol and water.

Certain embodiments of the invention provide a pharmaceutical composition suitable for topical administration that consists of more than about 5% (w/w) glycerol monolaurate (GML), polysorbate 80, hydroxyethyl cellulose, glycerol and water.

### Methods of Making a Composition Described Herein

Certain embodiments of the invention provide a method of preparing a glycerol monolaurate (GML) cream, comprising combining GML and a surfactant to provide a first mixture; combining a thickening agent, an alcohol and water to provide a second mixture; and combining the first and second mixtures under conditions suitable to provide a cream.

In certain embodiments, the conditions suitable to provide a cream comprise mixing.

In certain embodiments, the first mixture is heated prior to being combined with the second mixture (e.g., so as first mixture becomes a first solution). In certain embodiments, the second mixture is heated prior to being combined with the first mixture (e.g., so as second mixture becomes a second solution). In certain embodiments, the first and second mixtures are heated prior to being combined. In certain embodiments, the first and/or second mixture is heated to at least about, e.g., 60° C., 65° C., 70° C., 75° C. or 80° C. In certain embodiments, the first and/or second mixture is heated to about 70° C. In certain embodiments, the mixture(s) is/are heated in a water bath.

In certain embodiments, the GML, surfactant, thickening agent and/or alcohol is used in any weight percent described herein.

In certain embodiments, the surfactant is polysorbate 80 or any other surfactant described herein.

In certain embodiments, the thickening agent is hydroxyethyl cellulose (HEC) or any other thickening agent described herein.

In certain embodiments, the alcohol is glycerol or any other alcohol described herein.

Certain embodiments of the invention provide a glycerol monolaurate (GML) cream prepared by a method described herein.

### Methods of Use

Certain embodiments of the invention provide a method for delivering glycerol monolaurate (GML) to a mammal in need thereof, comprising topically administering an effective amount of a pharmaceutical composition as described herein to the mammal.

As used herein, the term “a mammal in need thereof”, refers to a mammal (e.g., a human, e.g., a female human) that is at a higher risk of contracting human immunodeficiency virus (HIV). Such mammals, include, e.g., women who live in regions with a high incidence of HIV, such as women living in sub-Saharan Africa, India and the U.S., as well as women who engage in activities that put them at higher risk (e.g., are sexually active, have multiple sexual partners or are sex workers). Accordingly, in certain embodiments, the administration reduces the likelihood of HIV transmission. In certain embodiments, the administration reduces the likelihood of HIV transmission by at least about, e.g., 20%, 30%, 40%, 50%, 60%, 70%, 80%, 90%, or more.

The phrase “effective amount” means an amount of a pharmaceutical composition described herein, that reduces the likelihood of HIV transmission.

The term “mammal” as used herein refers to humans, higher non-human primates, rodents, domestic, cows, horses, pigs, sheep, dogs and cats. In certain embodiments, the mammal is a female mammal. In one embodiment, the mammal is a human. In one embodiment, the mammal is a female human.

In certain embodiments, the pharmaceutical composition is administered vaginally.

In certain embodiments, the pharmaceutical composition is administered daily.

In certain embodiments, the method further comprises administering at least one other therapeutic agent to the mammal. In certain embodiments, the at least one other therapeutic agent is an agent that reduces the likelihood of HIV transmission. In certain embodiments, the at least one other therapeutic agent is an antiretroviral agent (e.g., tenofovir alafenamide fumarate, tenofovir disoproxil fumarate, tenofovir, cabotegravir or salts thereof, or rilpivirine or salts thereof).

Certain embodiments of the invention provide a pharmaceutical composition described herein for use in medical therapy.

Certain embodiments of the invention provide a pharmaceutical composition described herein for reducing the likelihood of human immunodeficiency virus (HIV) transmission.

Certain embodiments of the invention provide the use of a pharmaceutical composition described herein to prepare a medicament for reducing the likelihood of human immunodeficiency virus (HIV) transmission in a mammal (e.g., a human).

### Administration

A pharmaceutical composition described herein may be topically administered to a mammalian host, such as a human patient.

Useful dosages of a pharmaceutical composition described herein can be determined by comparing its in vitro activity, and in vivo activity in animal models. Methods for the extrapolation of effective dosages in mice, and other animals, to humans are known to the art; for example, see U.S. Pat. No. 4,938,949.

The amount of the pharmaceutical composition required for use in treatment will vary with the age and condition of the patient and will be ultimately at the discretion of the attendant physician or clinician. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

A pharmaceutical composition of the invention can also be administered in combination with other therapeutic agents, for example, other agents that are useful for reducing HIV transmission. Examples of such agents include antiretroviral agents (e.g., tenofovir alafenamide fumarate, tenofovir disoproxil fumarate, tenofovir, cabotegravir or salts thereof, or rilpivirine or salts thereof). Accordingly, in one embodiment the invention a pharmaceutical composition described herein may further comprise at least one other therapeutic agent.

The invention also provides a kit comprising a pharmaceutical composition described herein, packaging material, and instructions for administering the pharmaceutical composition to a mammal reduce the likelihood of HIV transmission. In certain embodiment, the kit further comprises at least one other therapeutic agent useful for reducing the likelihood of HIV transmission (e.g., an antiretroviral agent).

Some additional non-limiting embodiments are provided below to further exemplify the present disclosure:

1. A pharmaceutical composition suitable for topical administration that comprises more than 5% (w/w) glycerol monolaurate (GML).
2. The pharmaceutical composition of embodiment 1, that comprises at least about 10% (w/w) GML.
3. The pharmaceutical composition of embodiment 1, that comprises at least about 20% (w/w) GML.
4. The pharmaceutical composition of embodiment 1, that comprises at least about 30% (w/w) GML.
5. The pharmaceutical composition of embodiment 1, that comprises at least about 35% (w/w) GML.
6. The pharmaceutical composition of any one of embodiment 1-5, further comprising a thickening agent.
7. The pharmaceutical composition of embodiment 6, that comprises between about 0.1% to about 5% (w/w) thickening agent.
8. The pharmaceutical composition of embodiment 6, that comprises between about 0.5% to about 1.5% (w/w) thickening agent.
9. The pharmaceutical composition of embodiment 6, that comprises about 1% (w/w) thickening agent.
10. The pharmaceutical composition of any one of embodiments 6-9, wherein the thickening agent is hydroxyethyl cellulose (HEC).
11. The pharmaceutical composition of any one of embodiments 1-10, further comprising a surfactant.
12. The pharmaceutical composition of embodiment 11, that comprises between about 1% to about 5% (w/w) surfactant.
13. The pharmaceutical composition of embodiment 11, that comprises between about 1% to about 3% (w/w) surfactant.
14. The pharmaceutical composition of embodiment 11, that comprises about 2% (w/w) surfactant.
15. The pharmaceutical composition of any one of embodiments 11-14, wherein the surfactant is polysorbate 80.
16. The pharmaceutical composition of any one of embodiments 1-15, further comprising an alcohol.
17. The pharmaceutical composition of embodiment 16, that comprises between about 1% to about 40% (w/w) alcohol.
18. The pharmaceutical composition of embodiment 16, that comprises between about 10% to about 30% (w/w) alcohol.
19. The pharmaceutical composition of embodiment 16, that comprises about 20% (w/w) alcohol.
20. The pharmaceutical composition of any one of embodiments 16-19, wherein the alcohol is glycerol.
21. The pharmaceutical composition of any one of embodiments 1-20, further comprising water.
22. A pharmaceutical composition suitable for topical administration that comprises

a) more than about 5% (w/w) glycerol monolaurate (GML);

b) about 2% (w/w) polysorbate 80;

c) about 1% (w/w) hydroxyethyl cellulose (HEC);

d) about 20% (w/w) glycerol; and

e) water.

23. A method of preparing a glycerol monolaurate (GML) cream, comprising:

combining GML and a surfactant to provide a first mixture;

combining a thickening agent, an alcohol and water to provide a second mixture; and

combining the first and second mixtures under conditions suitable to provide a cream.

24. The method of embodiment 23, wherein the surfactant is polysorbate 80.
25. The method of embodiment 23 or 24, wherein the thickening agent is hydroxyethyl cellulose (HEC).
26. The method of any one of embodiments 23-25, wherein the alcohol is glycerol.
27. The method of any one of embodiments 23-26, wherein the conditions suitable to provide a cream comprise mixing.
28. The method of any one of embodiments 23-27, wherein the first mixture is heated prior to being combined with the second mixture.
29. The method of any one of embodiments 23-28, wherein the second mixture is heated prior to being combined with the first mixture.
30. The method of any one of embodiments 28-29, wherein the first mixture and/or second mixture is heated to about 70° C.
31. A method to deliver glycerol monolaurate (GML) to a mammal in need thereof, comprising topically administering an effective amount of a pharmaceutical composition as described in any one of embodiments 1-22 to the mammal.
32. The method of embodiment 31, wherein the mammal is a female mammal.
33. The method of embodiment 32, wherein the pharmaceutical composition is administered vaginally.
34. The method of any one of embodiments 31-33, wherein the administration reduces the likelihood of human immunodeficiency virus (HIV) transmission.
35. A pharmaceutical composition as described in any one of embodiments 1-22 for use in medical therapy.
36. A pharmaceutical composition as described in any one of embodiments 1-22 for reducing the likelihood of human immunodeficiency virus (HIV) transmission.
37. The use of a pharmaceutical composition as described in any one of embodiment 1-22 to prepare a medicament for reducing the likelihood of human immunodeficiency virus (HIV) transmission in a mammal.
38. A kit comprising a pharmaceutical composition as described in any one of embodiments 1-22 and instructions for administering an effective amount of the pharmaceutical composition to a mammal in need thereof to reduce the likelihood of human immunodeficiency virus (HIV) transmission.
39. The kit of embodiment 38, further comprising at least one other therapeutic agent useful for reducing the likelihood of HIV transmission.
40. The kit of embodiment 39, wherein the at least one other therapeutic agent is an antiretroviral agent.

The invention will now be illustrated by the following non-limiting Example.

### Example 1. Evaluation of Vaginal Drug Levels and Safety of a Locally Administered Glycerol Monolaurate Cream in Rhesus Macaques

The human immunodeficiency virus (HIV) epidemic affects millions of people worldwide. As women are more vulnerable to infection, female-controlled interventions can help control the spread of the disease significantly. Glycerol monolaurate (GML), an inexpensive and safe compound, has been shown to protect against simian immunodeficiency virus infection when applied vaginally. However, due to its low aqueous solubility, fabrication of high dose formulations of GML has proven difficult. Described herein is the development of a vaginal cream that could be loaded with up to 35% GML. Vaginal drug levels and safety of three formulations containing increasing concentrations of GML (5, 15 and 35% w/w) were tested in rhesus macaques following vaginal administration. GML concentration in the vaginal tissue increased as the drug concentration in the cream increased, with 35% GML cream resulting in tissue concentration of ˜0.5 mg/g, albeit with high inter-individual variability. Compared to the vehicle control, none of the GML creams had any significant effect on the vaginal flora and cytokine (MIP3α and IL-8) levels, suggesting that high dose GML formulations do not induce local adverse effects. In summary, the development of a highly-loaded vaginal cream of GML is described, along with vaginal drug levels and safety following local administration in macaques.

### Materials and Methods

### Materials

Tween® 80, glycerol, N,O-Bis(trimethylsilyl)trifluoroacetamide (BSTFA), pyridine and heptadecanoic acid were obtained from Sigma Aldrich (MO, USA). HyClone™ cell culture grade water was purchased from Thermo Scientific (MA, USA). GML was obtained from BASF Corporation (NJ, USA). Natrocellulose® HEC was obtained from Ashland Inc. (KY, USA).

### Preparation of GML Cream

To prepare GML cream, GML and Tween® 80 were weighed and transferred to a glass vial. HEC, glycerol and HyClone™ cell culture grade water were mixed in a separate glass vial. Both mixtures were heated to 70° C. for 10 minutes in a water bath. The cream was formed by adding the oil phase to the aqueous phase and mixing thoroughly.

For viscosity measurements, the cream was stored overnight at room temperature. For animal studies, the hot emulsion was filled into 1 mL needleless tuberculin syringes and allowed to cool in situ. The composition of the cream used for animal studies is listed in Table 1 below.

TABLE 1
Formulation of GML cream

| | Quantity |
|---|---|
| Ingredient | (% w/w) |
| Glycerol monolaurate | 5-35 |
| Tween 80 | 2 |
| Hydroxyethyl cellulose | 1 |
| Glycerol | 20 |
| Water | q.s. |

### Rheology of GML Creams

To determine the influence of HEC on the viscosity of the cream, the quantity of the polymer was varied from 0.5-2.5% w/w while maintaining the concentration of GML constant (15% w/w). To determine the influence of GML concentration on the viscosity, the quantity of GML was varied from 5-15% w/w at a HEC concentration of 2.5% w/w.

Rheometry was performed using an Ares-G2 parallel plate rheometer (plate diameter 25 mm, 25° C.). An appropriate amount of cream was loaded on to the plate using a spatula. The shear rate was increased as a step function and viscosity was measured at each shear rate. Power law was used to model the relationship between viscosity and shear rate (Berg J C. 2010. An Introduction to Interaces and Colloids: The Bridge to Nanoscience. ed.: World Scientific).

η=K*γⁿ⁻¹
η=viscosity
γ=shear rate
K=flow consistency index
n=flow behavior index

The values of K and n were used to determine the intrinsic viscosity and pseudoplastic behavior of the system, respectively.

### Rhesus Macaques, Product Administration and Collection of Samples

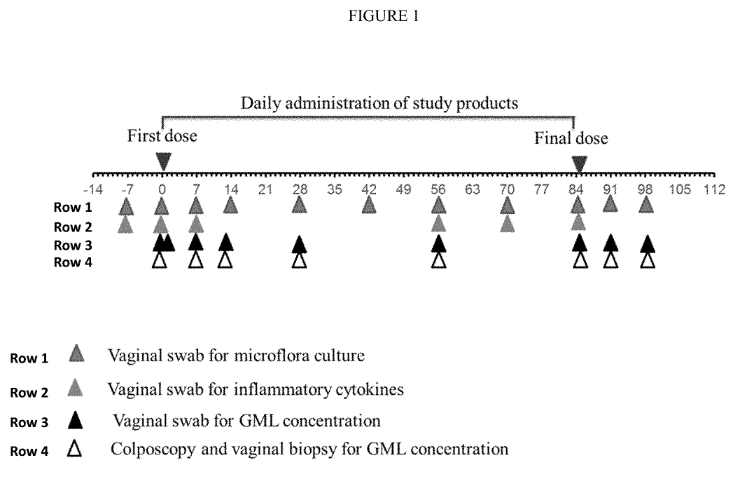

The National Research Council Guide for the Care and Use of Laboratory Animals was followed and from the University of Wisconsin Institutional Animal Care and Use Committee (IACUC) was received for this study. Twelve female rhesus macaques (5 to 12 years old) were randomly assigned to one of the four study groups: HEC/Tween® 80, 5% GML, 15% GML or 35% GML. Macaques were housed in pairs in standard stainless steel primate cages (Suburban Surgical, Chicago, Ill.). Care of the animals was performed as previously described (Schlievert et al. 2008. Antimicrob Agents Chemother 52(12):4448-4454). For product administration and collection of swabs and biopsies, animals were transferred to a table top restraint device using a transfer box and were gently restrained. Prior to sample collection or dosing, the genital region of each animal was wiped with dilute chlorhexidine solution in a single motion from vagina to anus followed by a clean gauze wipe in the same direction. The vagina was manually opened slightly, and a 1 mL syringe without a needle was inserted atraumatically into the vagina until approximately the 0.4 mL mark. A dose of administered agent was delivered into the vagina and the syringe was removed; animals were dosed once a day, between 7:00 a.m. and 8:00 a.m. Control animals received 1 mL of vehicle control gel, and GML animals received 1 mL of GML containing gel. For collection of vaginal swabs for microbial and cytokine studies, swabs remained in the vagina for approximately 1 min before they were carefully removed and placed in appropriate containers. Colposcopy was performed prior to biopsy to evaluate for gross inflammation. Findings were graded as previously described (Schlievert et al. 2008. Antimicrob Agents Chemother 52(12):4448-4454). Animals were anesthetized for cervical biopsies, which were performed as previously described. Cervical biopsy samples for drug analysis were placed in 1.5 mL Eppendorf tubes, and shipped priority overnight on dry ice to the University of Minnesota, Minneapolis, Minn. Schedule and timing of sample collection is detailed in FIG. 1 Determination of GML concentrations in tissue biopsies and swabs

GML was extracted from tissue biopsies and swabs using dichloromethane for 2 h at room temperature. A fraction of the dichloromethane extract was transferred to vial inserts (Sigma Aldrich, USA) and spiked with the internal standard (heptadecanoic acid dissolved in dichloromethane). The solvent was evaporated under nitrogen gas for 30 minutes at room temperature. The samples were reconstituted in a mixture of BSTFA and pyridine (5:1 v/v) and placed in a pre-heated oven at 70° C. for 30 minutes. The samples were cooled to room temperature and analyzed by GC/MS.

For GC/MS analysis, 2 μL of sample was injected onto a DB5 column (30 m×0.25 mm ID, 0.25 μm film thickness). The temperatures of the injector and detector were maintained at 300° C. The initial column temperature was 100° C. After 1 min, the column temperature was increased to 150° C. at a rate of 10° C./min, and then increased to 210° C. at a rate of 25° C./min. Finally, it was increased to 320° C. at a rate of 35° C./min and held there for 7.1 minutes. The total run time was 18.64 minutes.

Ions were generated using electron impact ionization. Two ions were monitored—GML (m/z 315.2) and heptadecanoic acid (m/z 327.2). The retention times were 9.42 min and 9.22 min respectively.

### Cytokine Analysis

Concentrations of interleukin 8 (IL-8) and macrophage inflammatory protein 3α (MIP3α) in cervical vaginal fluid (CVF) were measured at two points prior to product administration (day −7 and day 0) and at weeks 1, 8, 10 and 12 during the period of daily product administration (FIG. 1). CVF was collected using weighed cotton swabs that consistently and reproducibly absorbed 0.1 mL of CVF. CVF was always collected before administration of the vaginal product. After collection, swabs were placed at 4° C. and shipped from the Wisconsin National Primate Research Center to the University of Minnesota. Upon arrival, swabs were diluted in 0.9 mL of phosphate buffered saline and concentration of MIP3α and IL-8 was measured by ELISA according to the manufacturer's directions (R&D Systems, Minneapolis, Minn.).

### Vaginal Microflora Determination

CVF for microflora determination was collected on swabs and shipped to the University of Minnesota as described above for cytokine samples. Samples were obtained 7 days prior to product administration, the day of treatment initiation and weeks 1, 2, 4, 6, 8, 10 and 12 during the period of daily product administration. Swabs were also collected 1 and 2 weeks after the last dose of vaginal products were administered (FIG. 1). Upon arrival to the University of Minnesota, swabs were placed in 0.9 mL pre-cooled Todd-Hewitt broth at 4° C. and then serially diluted in additional broth for quantitative counts on chocolate agar plates that were incubated aerobically at 37° C. in a 7% CO₂ incubator. Lactobacilli, staphylococci, streptococci, gram-negative rods and yeasts were identified using methods previously described (Schlievert et al. 2008. Antimicrob Agents Chemother 52(12):4448-4454).

### Statistical Analysis

Statistical analyses were performed using one-way analysis of variance (ANOVA) and post-hoc Bonferroni test. Differences were considered statistically significant if p<0.05.

### Results

### Rheology of GML Creams

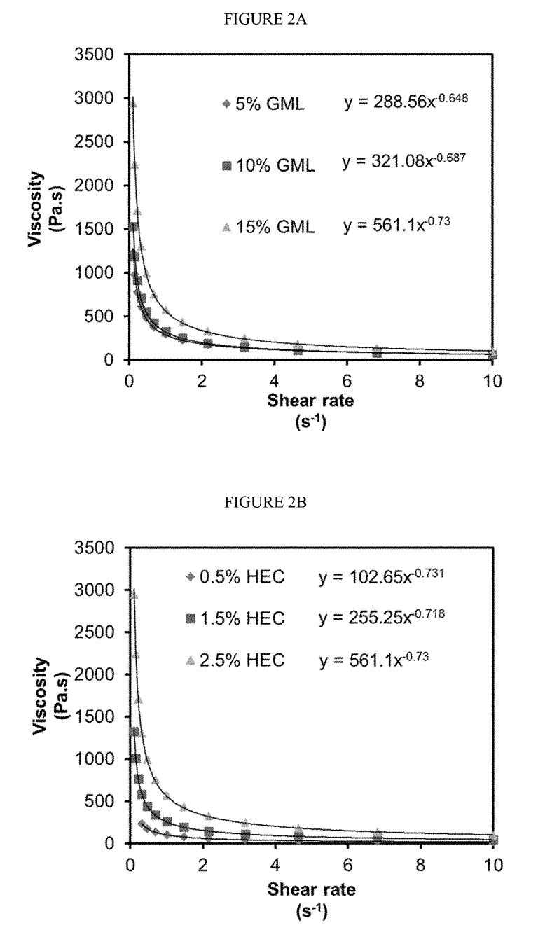

The viscosity of GML creams was analyzed using a plate rheometer. FIG. 2A shows the effect of concentration of GML on the viscosity of the cream. All formulations displayed a shear thinning behavior. The flow consistency index increased with an increase in GML concentration. The flow behavior index decreased slightly with an increase in concentration of GML.

The effect of different concentrations of HEC on the rheology of the cream is shown in FIG. 2B. As noted with increasing concentrations of GML, with an increase in concentration of HEC there was an increase in the flow consistency index. This indicated that the creams with higher levels of GML and HEC had a higher viscosity. However, the flow behavior index was unaffected by changes in the concentration of HEC. This indicated that the pseudoplastic behavior of the cream was unaffected by HEC concentration.

The effect of GML and HEC concentrations on the rheology of GML creams is summarized in Table 2.

TABLE 2
Effect of GML and HEC concentration on the rheological properties of
GML cream

| Concentration of | Concentration of | Flow consistency | |
|---|---|---|---|
| GML | HEC | index, K | Flow behavior |
| (% w/w) | (% w/w) | (Pa · s) | index, n |
| 5 | 2.5 | 288.56 | 0.352 |
| 10 | 2.5 | 321.08 | 0.313 |
| 15 | 2.5 | 561.1 | 0.27 |
| 15 | 1.5 | 255.25 | 0.282 |
| 15 | 0.5 | 102.65 | 0.269 |

### Vaginal Levels of GML in Rhesus Macaques Following Local Delivery

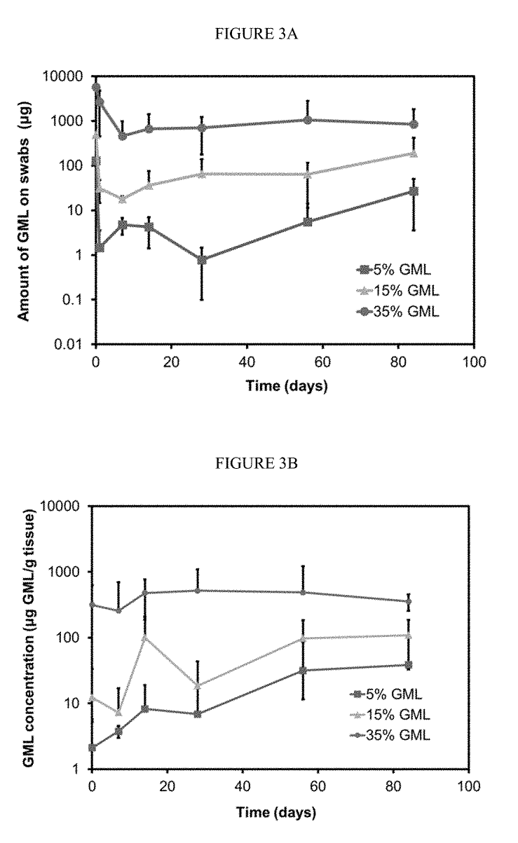

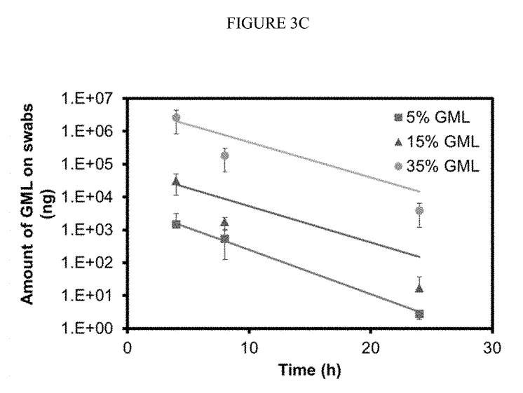

The levels of GML in tissue biopsies and swabs 4 h post dose at various times was monitored during the treatment cycle (FIG. 3A). As expected, GML concentrations on swabs were highest in animals treated with 35% GML cream. On days 0 and 1, the levels of GML on the swabs were higher than those observed on the later days. The levels of GML remained constant over the rest of the study. The levels of GML on the swabs at day 7 and 14 was also monitored after discontinuation of the treatment. GML was undetectable in all the treatment groups.

The concentration of GML in vaginal tissue biopsies was measured during the treatment cycle. Similar to the results with swabs, with an increase in concentration of GML in the cream there was an increase in GML concentration in the tissue biopsies (FIG. 3B). However, no detectable levels of GML were observed at 7 and 14 days after the treatment was stopped.

Analysis of the GML levels on swabs and in biopsies with the different formulations is shown in Table 3.

TABLE 3
Analysis of vaginal uptake of GML

| | Formulations | | |
|---|---|---|---|
| | 5% GML | 15% GML | 35% GML |
| Swabs | | | |
| Maximum observed amount (μg) | 128 ± 113 | 495 ± 206 | 5705 ± 3774 |
| Time at maximum amount (days) | 0 | 0 | 0 |
| AUC₀⁸⁴ (μg * days/g) | 696 | 6641 | 78018 |
| Biopsies | | | |
| Maximum observed | 39 ± 63 | 109 ± 76 | 518 ± 581 |
| concentration (μg/g) | | | |
| Time at maximum | 84 | 84 | 28 |
| concentration (days) | | | |
| AUC₀⁸⁴ (μg * days/g) | 1823 | 6169 | 38574 |

To determine the clearance kinetics of GML from the vaginal cavity, the amount of GML on swabs obtained 4, 8 and 12 h post dose was monitored. These results are shown in FIG. 3C. The elimination of GML from the vaginal cavity followed a first order rate. A curve describing first order kinetics was fit to the data to estimate the rate constant of elimination of GML. The elimination rate constant was found to independent of the concentration of GML in the cream (0.25-0.3 h⁻¹). These results are summarized in Table 4.

TABLE 4
Elimination kinetics of GML delivered in various formulations

| Concentration | Elimination | |
|---|---|---|
| of GML | rate constant | Amount at time 0 |
| (% w/w) | (h⁻¹) | (μg) |
| 5 | 0.31 | 5.3 |
| 15 | 0.25 | 66.5 |
| 35 | 0.25 | 5477 |

### Cytokine Analysis

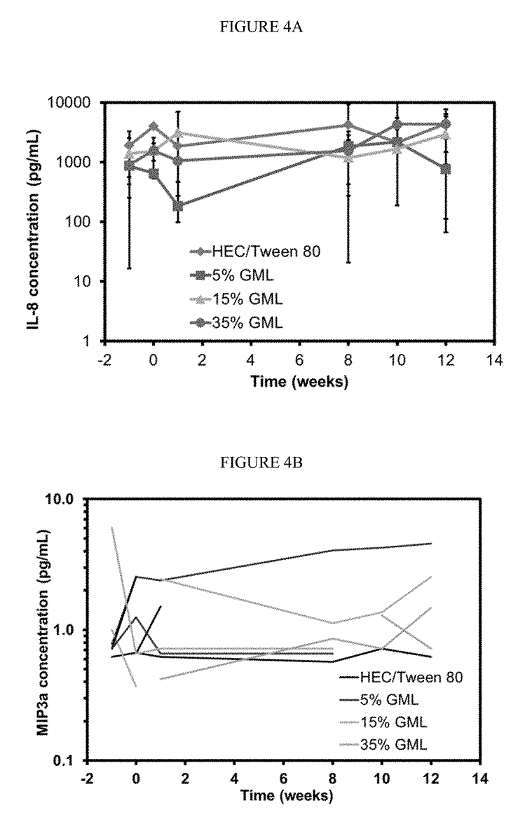

Overall, there was no difference in concentration of IL-8 in CVF in GML treated groups compared to HEC treated animals (FIG. 4A). However, the HEC treated group had a higher level of IL-8 at baseline (day 0; prior to administration of study products) than GML treated animals (p<0.05). No statistically significant difference was seen at any other time point. Although concentrations of MIP3α were quite variable in all groups throughout the study period, there was no overall difference between HEC and GML treated groups and no difference in MIP3a concentration at any single time point (FIG. 4B).

### Vaginal Microflora Determination

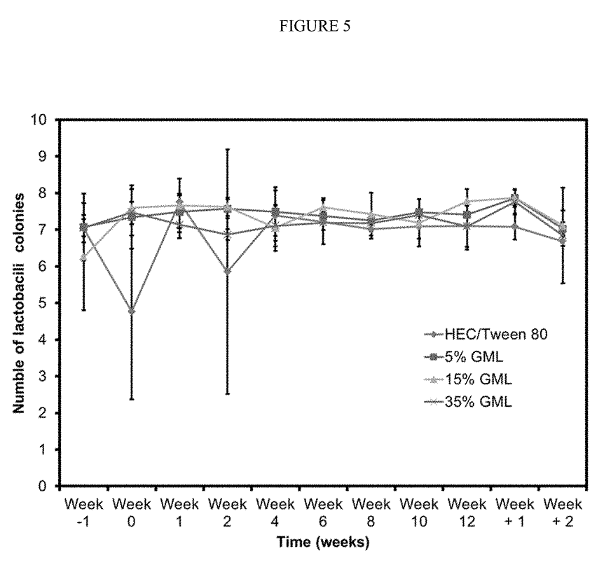

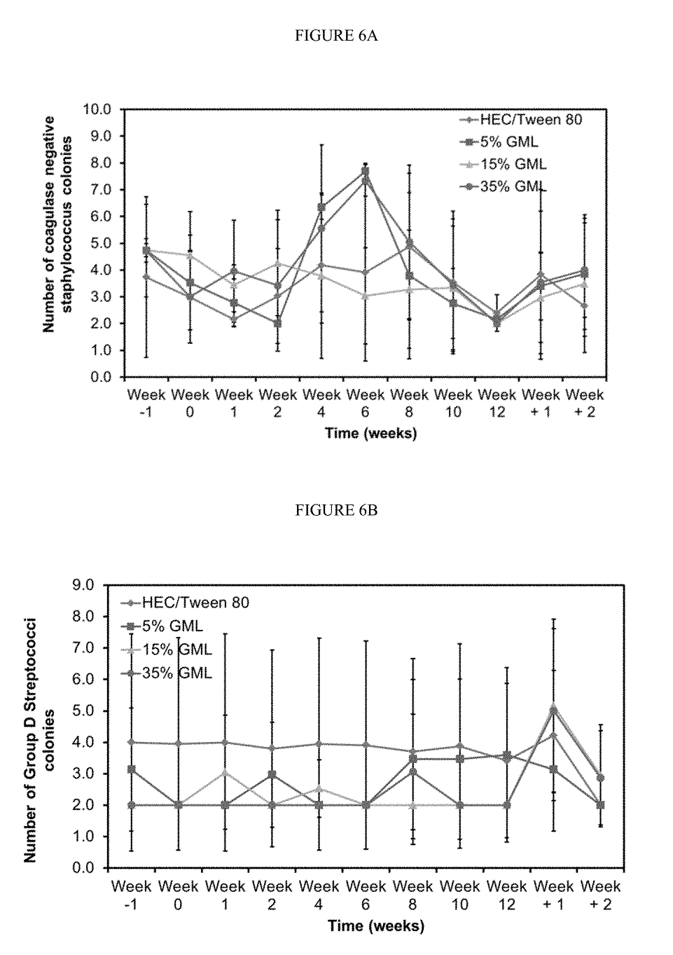

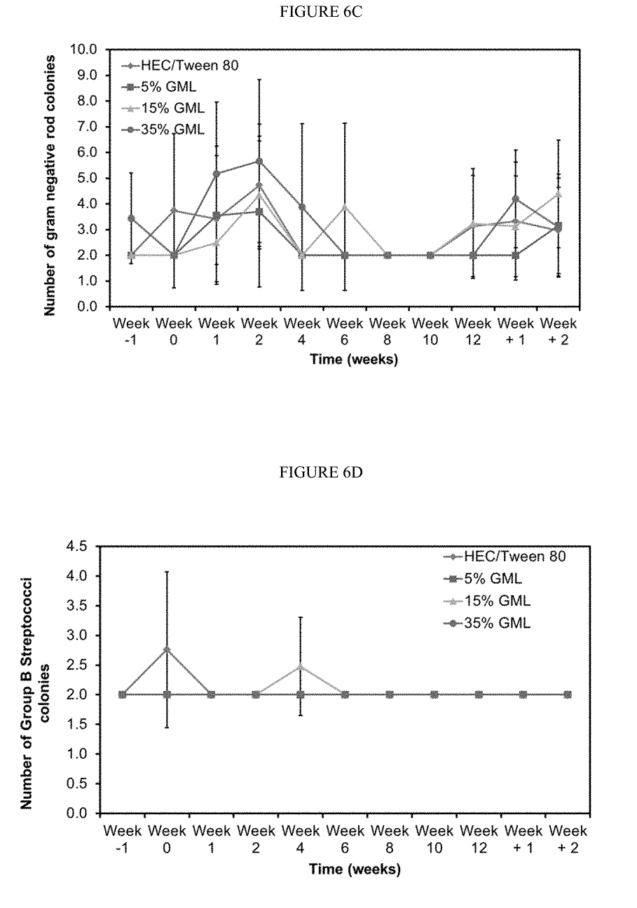

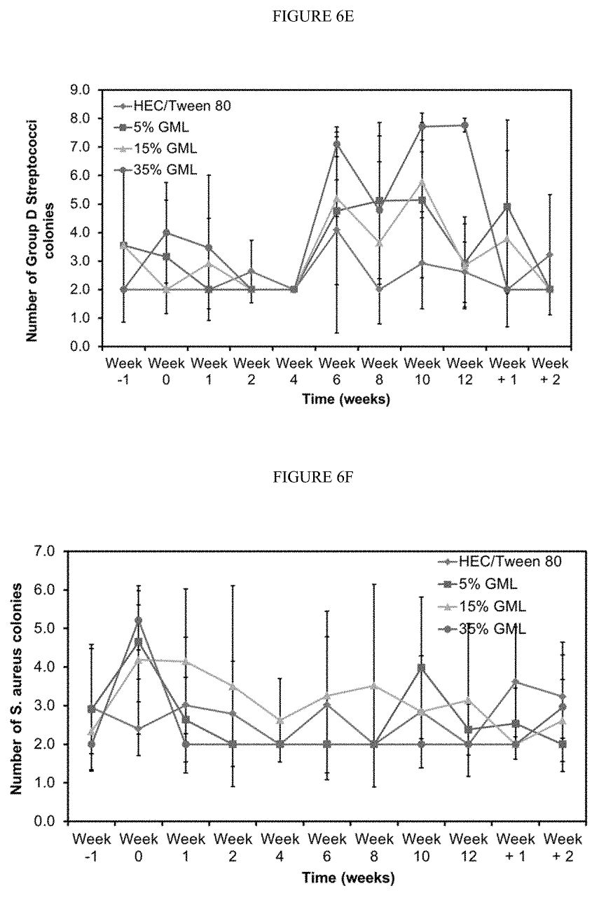

In all animals, Lactobacillus was the most commonly isolated organism. Number of Lactobacilli colonies remained relatively constant over the entire study period in all animals (FIG. 5). There were fewer Lactobacilli isolated from HEC treated animals compared to GML treated animals one week after completion of the vaginal product treatment course (p=<0.05) but no difference at any other time point and no overall difference between GML treated and HEC treated animals. Except for two time points (week 10 and 12) in animals treated with 35% GML cream, there were no significant differences in the levels of other organisms (FIGS. 6A-F).

### Colposcopy

Colposcopy was performed prior to the collection of biopsies. No abnormalities were identified in any of the animals assigned to the HEC/Tween or 5% GML groups. One of animals receiving 15% GML was noted to have mild epithelial disruption and cervical hyperemia at week 12 but no abnormal findings before or after. The other 2 animals in this treatment arm had normal exams at all time points. In the 35% GML treatment arm, one animal had mild inflammation, cervical hyperemia and epithelial disruption at week 8 only. The other 2 animals had no abnormalities.

Significant gel leakage was noted in 2 of the HEC treated animals and 2 of the 5% GML treated animals. Mild intermittent leakage was noted in 2 of the 15% GML treated animals and in none of the 35% treated animals.

### Discussion

HIV infection remains a major health concern, particularly in many African countries. The use of vaginal products as a preventive for HIV transmission has great potential for containing the spread of this disease (Lederman et al. 2006. Nat Rev Immunol 6(5):371-382). GML is an affordable and safe anti-microbial that has been shown to be highly effective at preventing the vaginal transmission of SIV. However, the GML formulation that was used in microbicide studies to date is not optimal from a pharmacologic standpoint given issues obtaining concentrations of GML over 5%, the need to use a hydrophobic vehicle and its low viscosity which contributes to significant leakiness. The goal was to develop an acceptable vaginal formulation of GML capable of delivering high doses in an aqueous vehicle.

HEC gels are considered the ‘universal placebo’ for vaginal microbicide trials given their stability, lack of epithelial toxicity and lack of anti-HIV activity (Tien et al. 2005. AIDS Res Hum Retroviruses 21(10):845-853; Richardson et al. 2013. J Acquir Immune Defic Syndr 63(1):120-125). However, aqueous gels are not suitable for the delivery of hydrophobic molecules. This is particularly problematic for high dose drugs such as GML. To overcome this issue, an emulsion-based method to incorporate GML in HEC gels was developed. This technology allowed for the incorporation of up to 35% w/w of GML in the formulation. The formulation consisted of all FDA-approved excipients and involved simple manufacturing conditions.

Further, it was hypothesized that a higher viscosity of a cream formulation would enable a longer residence time for GML. Administering GML in highly loaded GML creams helped achieve higher local concentrations of the cream in the vaginal tract. The concentration of GML achieved with 35% cream remained higher than the initial concentration achieved by 5% creams for at least 24 hours. This indicates that 35% GML creams may provide protection similar to the 5% creams for a longer period of time, thus decreasing the frequency of dosing. Reducing dosing frequency may positively impact patient adherence to the product (Chesney MA 2000. Clin Infect Dis 30 Suppl 2:S171-176). It should be noted however that this effect is manifested by higher dose administered and not by a lower clearance. The elimination kinetics for the drug remained identical across the various concentrations in the cream. The residence time of the gel was generally limited by the turnover of fluids in the vaginal cavity (Sassi et al. 2008. J Pharm Sci 97(8):3123-3139). The use of mucoadhesive excipients and/or increasing the viscosity of the formulation may provide a means to further improve residence times.

In a blinded in vivo study, it was found that higher concentrations of GML in creams were associated with higher levels of the drug on swabs and in tissue biopsies. The amount of GML in tissue swabs and biopsies in animals treated with 15% GML creams were ˜3-fold higher than in animals treated with 5% GML. In contrast, amount of GML on vaginal swabs in animals treated with 35% GML creams was ˜20-50-fold higher than that in animals treated with 5% GML. The elimination rate constant for the drug was independent of drug loading in the cream (FIG. 3C). It is likely that the higher viscosity of 35% GML creams aids greater adhesion of the cream to the vaginal tract. This could explain why the initial concentration of GML achieved at the time of administration is disproportionately higher for the 35% GML cream (Table 4).

Previous studies have elaborated on the mechanism of action of GML (Li et al. 2009. Nature 458(7241):1034-1038; Haase AT 2011. Annu Rev Med 62:127-139). Exposure of the endocervical epithelium to simian immunodeficiency virus (SIV) initiates innate immune response. This provides the virus with a founder population of immune cells, which enables its systemic dissemination. GML blocks this initial signaling originating from the endocervical epithelium, thereby reducing the number of immune cells available to the virus. Thus, high local concentrations of GML are required for its activity. In this study, high local tissue concentrations were achieved by increasing the loading of the drug in the cream.

Despite significant standardization in the methods of collection of swabs and biopsies, there was significant inter-individual variability in GML levels. Clinical studies with vaginal rings point to differences in variable drug levels in different parts of the vaginal tract (Romano et al. 2009. AIDS Res Hum Retroviruses 25(5):483-488). Consequently, small variations in the site of biopsy could affect the observed tissue concentration. Additionally, differences in the turnover rate of vaginal fluids could significantly affect elimination rates. Due to high inter-individual variability, differences in concentrations observed with various formulations of GML were not statistically significant. Additional studies may be performed to establish these differences.

An additional issue with vaginal gel or cream formulations is the lack of patient adherence (Kirtane et al. 2016. J Pharm Sci 105(12):3471-3482). This may stem from poor physical properties of these formulations. For example, women using vaginal gels have reported leakage from the vaginal cavity and messiness associated with the use of these products (Rohan L C, Sassi A B 2009. AAPS J 11(1):78-87; Malonza et al. 2005. AIDS 19(18):2157-2163; Bentley et al. 2004. Am J Public Health 94(7):1159-1164). Additionally, due to the high turnover rate of vaginal fluids, the active drug has a low residence time at the site of administration and hence requires daily dosing (Rohan L C, Sassi A B 2009. AAPS J 11(1):78-87). It was reasoned that increasing the viscosity of these formulations, while maintaining pseudoplastic behavior, might help overcome both these issues. Incorporation of GML, a waxy hydrophobic compound, significantly increased the viscosity of the formulation, while minimally affecting its shear thinning properties. In fact, significantly less leakiness was noted in the new formulations compared to the original GML/KY warming formulation and, among the new formulations, leakiness improved as GML concentration increased. It is expected that these favorable rheological properties will improve patient acceptance of these formulations.

Effective clinical translation of this strategy may require chronic dosing of high amounts of GML. Hence, safety and tolerability of the formulation are of primary concern. Local delivery of GML achieved with this formulation does ensure that highest concentrations of GML are achieved at the site of action. The levels of two cytokines, MIP3α and IL-8, markers of inflammation, remained unaltered during the entire study. Additionally, these levels were comparable to the levels obtained with placebo treatment. Moreover, GML did not impact the levels of healthy lactobacilli in the CVF and no persistent signs of vaginal or cervical inflammation were seen in any of study animals.

In summary, described herein is the formulation, rheological analysis and vaginal concentrations of a novel, high-dose formulation of GML. The creams displayed pseudoplastic behavior. Topical application of the cream led to high concentrations in vaginal tissue biopsies (15-400 μg/g) with no detectable signs of inflammation or alteration of healthy vaginal microflora. Future studies may investigate the efficacy of these formulations of GML in preventing HIV transmission.

### CONCLUSION

The formulation of a highly loaded HEC based cream of GML is reported herein. Administration of the cream in the vaginal cavity was found to be safe and resulted in high GML concentrations in the vaginal cavity.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A pharmaceutical composition suitable for topical administration that comprises more than 5% (w/w) glycerol monolaurate (GML).

2. The pharmaceutical composition of claim 1, that comprises about 6-50% (w/w) GML.

3. The pharmaceutical composition of claim 1, further comprising a thickening agent.

4. The pharmaceutical composition of claim 3, that comprises between about 0.1% to about 5% (w/w) thickening agent.

5. The pharmaceutical composition of claim 3, wherein the thickening agent is hydroxyethyl cellulose (HEC).

6. The pharmaceutical composition of claim 1, further comprising a surfactant.

7. The pharmaceutical composition of claim 6, that comprises between about 1% to about 5% (w/w) surfactant.

8. The pharmaceutical composition of claim 6, wherein the surfactant is polysorbate 80.

9. The pharmaceutical composition of claim 1, further comprising an alcohol.

10. The pharmaceutical composition of claim 9, that comprises between about 1% to about 40% (w/w) alcohol.

11. The pharmaceutical composition of claim 9, wherein the alcohol is glycerol.

12. The pharmaceutical composition of claim 1, further comprising water.

13. A pharmaceutical composition suitable for topical administration that comprises:
a) more than about 5% (w/w) glycerol monolaurate (GML);
b) about 2% (w/w) polysorbate 80;
c) about 1% (w/w) hydroxyethyl cellulose (HEC);
d) about 20% (w/w) glycerol; and
e) water.

14. A method of preparing a glycerol monolaurate (GML) cream, comprising:
combining GML and a surfactant to provide a first mixture;
combining a thickening agent, an alcohol and water to provide a second mixture; and
combining the first and second mixtures under conditions suitable to provide a cream.

15. The method of claim 14, wherein the surfactant is polysorbate 80.

16. The method of claim 14, wherein the thickening agent is hydroxyethyl cellulose (HEC).

17. The method of claim 14, wherein the alcohol is glycerol.

18. A method of claim 14 further comprising vaginally administering an effective amount of the composition to a female mammal.

19. The method of claim 18, wherein the administration reduces the likelihood of human immunodeficiency virus (HIV) transmission.

20. The pharmaceutical composition of claim 13 for reducing the likelihood of human immunodeficiency virus (HIV) transmission.
